(19) United States
(12) Patent Application Publication
(10) Pub. No.: US 2023/0177242 A1
(43) Pub. Date: Jun. 8, 2023

(54) DIGITAL TWIN AIRFLOW MODELING

(71) Applicant: INTERNATIONAL BUSINESS MACHINES CORPORATION, ARMONK, NY (US)

(72) Inventors: Craig M. Trim, Ventura, CA (US); Nadiya Kochura, Bolton, MA (US); Fang Lu, Billerica, MA (US); Jeremy R. Fox, Georgetown, TX (US)

(21) Appl. No.: 17/643,086

(22) Filed: Dec. 7, 2021

(51) Int. Cl. G06F 30/28

(52) U.S. Cl. CPC G06F 30/28

(57) ABSTRACT

A method, computer system, and a computer program product for airflow modeling is provided. The present invention may include generating a digital twin for a structure of interest identified by a user. The present invention may include collecting data for one or more infectious diseases. The present invention may include performing a plurality of simulations of the digital twin for the one or more infectious diseases. The present invention may include providing one or more recommendations to the user based on at least the performance of the digital twin for the one or more infectious diseases.

### BACKGROUND

The present invention relates generally to the field of computing, and more particularly to digital twin airflow modeling.

Structures such as, but not limited to, office buildings, healthcare facilities, cruise ships, retail stores, and/or other structures may have the potential for at least, indoor air quality issues, illness transmission, occupational injuries, exposure to hazardous materials, and/or accidental falls. These potential hazards may require at least architects, engineers, and/or facility managers to design and/or maintain structures and/or processes which may help ensure occupant safety and/or health, by taking into consideration an infectious disease which may be present in the structure. Structure designs may focus on eliminating and/or preventing potential hazards to personnel, rather than relying on personal protective equipment and/or administrative procedures in preventing mishaps.

One potential method of eliminating and/or preventing potential hazards to personnel is airflow modeling. Airflow modeling may be utilized to simulate airflow in a structure which may enable at least architects, engineers, and/or facility managers in designing a structure and/or enhancing an existing structure such that air quality may be optimized.

### SUMMARY

Embodiments of the present invention disclose a method, computer system, and a computer program product for airflow modeling. The present invention may include generating a digital twin for a structure of interest identified by a user. The present invention may include collecting data for one or more infectious diseases. The present invention may include performing a plurality of simulations of the digital twin for the one or more infectious diseases. The present invention may include providing one or more recommendations to the user based on at least the performance of the digital twin for the one or more infectious diseases.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for airflow modeling according to at least one embodiment;

FIG. 3 is a block diagram of internal and external components of computers and servers depicted in FIG. 1 according to at least one embodiment;

FIG. 4 is a block diagram of an illustrative cloud computing environment including the computer system depicted in FIG. 1, in accordance with an embodiment of the present disclosure; and

FIG. 5 is a block diagram of functional layers of the illustrative cloud computing environment of FIG. 4, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for airflow modeling. As such, the present embodiment has the capacity to improve the technical field of digital twins by providing one or more recommendations to a user for the design and/or modification of a structure based on a plurality of simulations of a digital twin for one or more infectious diseases. More specifically, the present invention may include generating a digital twin for a structure of interest identified by a user. The present invention may include collecting data for one or more infectious diseases. The present invention may include performing a plurality of simulations of the digital twin for the one or more infectious diseases. The present invention may include providing one or more recommendations to the user based on at least the performance of the digital twin for the one or more infectious diseases.

As described previously, structures such as, but not limited to, office buildings, healthcare facilities, cruise ships, retail stores, and/or other structures may have the potential for at least, indoor air quality issues, illness transmission, occupational injuries, exposure to hazardous materials, and/or accidental falls. These potential hazards may require at least architects, engineers, and/or facility managers to design and/or maintain structures and/or processes which may help ensure occupant safety and/or health, by taking into consideration an infectious disease which may be present in the structure. Structure designs may focus on eliminating and/or preventing potential hazards to personnel, rather than relying on personal protective equipment and/or administrative procedures in preventing mishaps.

One potential method of eliminating and/or preventing potential hazards to personnel is airflow modeling. Airflow modeling may be utilized to simulate airflow in a structure which may enable at least architects, engineers, and/or facility managers in designing a structure and/or enhancing an existing structure such that air quality may be optimized.

Therefore, it may be advantageous to, among other things, generate a digital twin for a structure of interest identified by a user, collect data for one or more infectious diseases, perform a plurality of simulations of the digital twin for the one or more infectious diseases, and provide one or more recommendations to the user based on at least a performance of the digital twin in the plurality of simulations for the one or more infectious diseases.

According to at least one embodiment, the present invention may improve identifying ways to best design and/or modify a structure based on the plurality of simulations of the digital twin for the one or more infectious diseases.

According to at least one embodiment, the present invention may improve recommendations provided to a user based on the plurality of simulations by also considering user preferences which may include, but are not limited to including, cost of implementation, time frame for implementation, amongst other user preferences.

According to at least one embodiment, the present invention may improve identifying areas most critical to airflow by generating a heatmap. The heatmap may be integrated with the digital twin such that the digital twin may include one or more areas as to which areas of the structure may be most susceptible to microorganism accumulation.

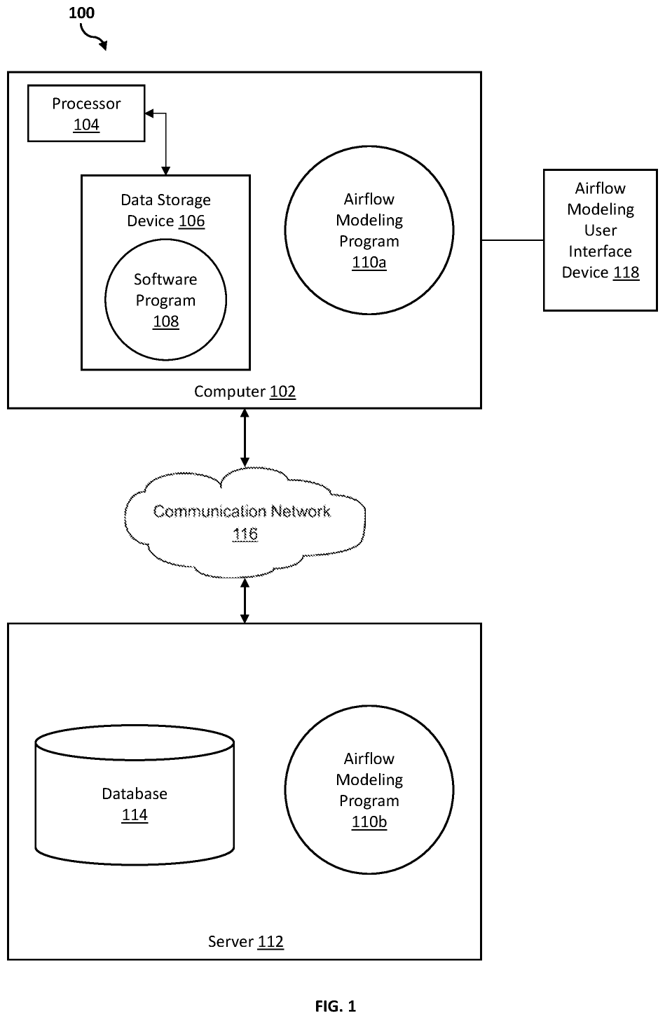

Referring to FIG. 1, an exemplary networked computer environment 100 in accordance with one embodiment is depicted. The networked computer environment 100 may include a computer 102 with a processor 104 and a data storage device 106 that is enabled to run a software program 108 and an airflow modeling program 110a. The networked computer environment 100 may also include a server 112 that is enabled to run an airflow modeling program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

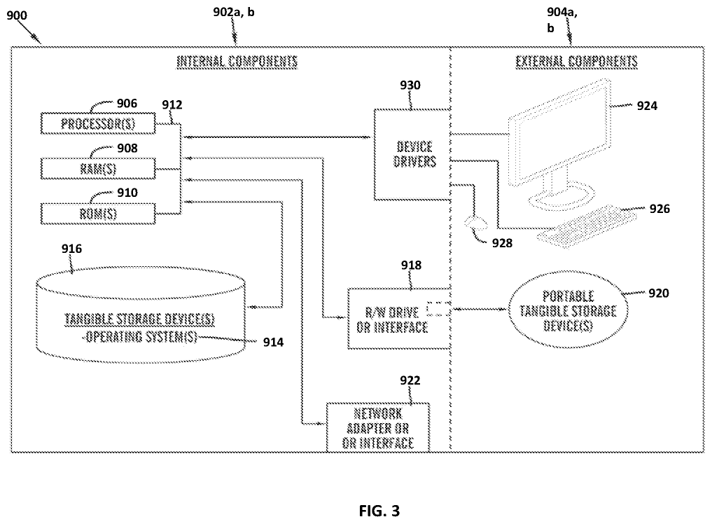

The client computer 102 may communicate with the server computer 112 via the communications network 116. The communications network 116 may include connections, such as wire, wireless communication links, or fiber optic cables. As will be discussed with reference to FIG. 3, server computer 112 may include internal components 902a and external components 904a, respectively, and client computer 102 may include internal components 902b and external components 904b, respectively. Server computer 112 may also operate in a cloud computing service model, such as Software as a Service (SaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the airflow modeling program 110a, 110b may interact with a database 114 that may be embedded in various storage devices, such as, but not limited to a computer/mobile device 102, a networked server 112, or a cloud storage service.

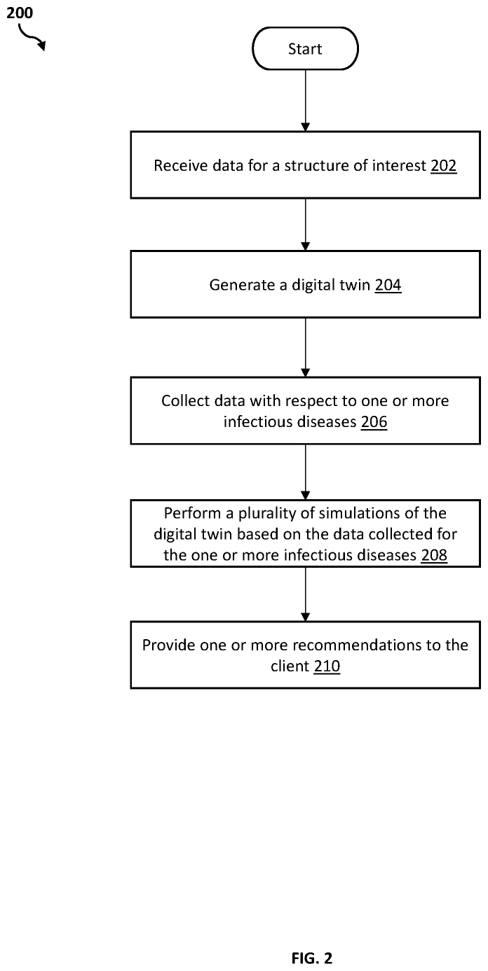

According to the present embodiment, a user using a client computer 102 or a server computer 112 may use the airflow modeling program 110a, 110b (respectively) to generate a digital twin for a structure of interest and simulate one or more infectious diseases. The airflow modeling method is explained in more detail below with respect to FIG. 2.

Referring now to FIG. 2, an operational flowchart illustrating the exemplary airflow modeling process 200 used by the airflow modeling program 110a and 110b (hereinafter airflow modeling program 110) according to at least one embodiment is depicted.

At 202, the airflow modeling program 110 receives data for a structure of interest. The structure of interest may be an office building, retail store, healthcare facility, cruise ship, warehouse, and/or other structure in which occupants may be at risk of potential hazards related to air quality. The structure of interest may be an existing structure and/or a design for a new structure. The structure of interest may be identified by a user in an airflow modeling user interface 118.

The airflow modeling program 110 may receive and/or access data with respect to the structure of interest identified by the user. The airflow modeling program 110 may receive and/or access data for the structure of interest from at least, the user, one or more Internet of Things (IoT) devices associated with the structure, and/or one or more publicly available resources, amongst other methods of receiving and/or accessing data. The airflow modeling program 110 may store the data received and/or accessed with respect to the structure in a knowledge corpus (e.g., database 114).

The user may provide data, such as, but not limited to, square footage, property size, location, material used in construction, window types, year built, blueprints, roofing details, architecture, information on appliances, occupancy, employee schedules, capacity, hours of operation, safety procedures, safety standards, amongst other data. As will be explained in more detail below with respect to step 206 below, the location and other data received by the airflow modeling program 110 may be leveraged in collecting data with respect to one or more infectious diseases. The user may provide data to the airflow modeling program 110 utilizing the airflow modeling user interface 118. The airflow modeling program 110 may also request data with respect to the structure of interest utilizing one or more prompts. The one or more prompts may be displayed to the user in the airflow modeling user interface 118 and may be generated based on the type of structure identified by the user in the airflow modeling user interface 118. As will be explained in more detail below with respect to step 208, the data received and/or accessed for the structure may be utilized in simulating the airflow within the structure with respect to one or more infectious diseases.

For example, if the structure of interest identified by the user in the airflow modeling user interface 118 is a healthcare facility, then the airflow modeling program 110 may display one or more prompts in the airflow modeling user interface 118 requesting data, such as, but not limited to, outflow of trash, outflow of recyclables, outflow of hazardous materials, patient intake numbers, capacity, electronic health records (EHRs) in instances where consent to disclose such records has been received, room and/or unit identification, amongst other data. In the example of the healthcare facility, the user may also integrate their patient intake system with the airflow modeling program 110, as will be explained in more detail below with respect to step 210. The airflow modeling program 110 may not receive, access, and/or integrate patient intake systems with respect to healthcare facilities and/or any other structure in violation of any local, state, federal, or international law with respect to data privacy protection. The airflow modeling program 110 may provide one or more operational recommendations to the user based on patient intake, such as, but not limited to, room assignments, room rotations, identifying patient capacity thresholds, determining time periods in which a room may be utilized for a new patient based on a diagnosis of a previous patient, sanitation scheduling, amongst other operational recommendations. The airflow modeling program 110 may not receive and/or access any data in violation of any local, state, federal, or international law with respect to privacy protection. In this example, the airflow modeling program 110 may request consent prior to displaying prompts and/or accessing data related to EHRs, patient intake data, and/or other data.

The airflow modeling program 110 may also access and/or receive air quality requirements based on at least the structure and/or location of the structure. The air quality requirements may be provided by the user, for example, the user may provide internal safety requirement documents and/or the airflow modeling program 110 may access air quality requirements based on at least the type of structure and/or the location of the structure. As will be explained in more detail below, the airflow modeling program 110 may provide one or more recommendations to the user in the airflow modeling user interface 118 based on air quality requirements.

The airflow modeling program 110 may also receive and/or access data from one or more IoT devices utilized in controlling and/or monitoring the environment of the structure. IoT devices may control and/or monitor, but are not limited to controlling and/or monitoring, thermostats, lighting, air quality, smoke detectors, carbon monoxide detectors, irrigation systems, air conditioning, movement, and/or ventilation systems. The one or more IoT devices may monitor the environment of the structure by performing readings of the of the environment of the structure. The one or more IoT devices may be connected to at least one sensor (e.g., temperature sensor, motion sensor, humidity sensor, pressure sensor, accelerometer, gas sensor, multi-purpose IoT sensor, among other sensors) to perform the reading. The IoT device may store data with respect to the reading on the IoT device. The IoT device may broadcast the data to a comprehensive database. The comprehensive database may be a shared ledger (e.g., distributed ledger, hyperledger). The airflow modeling program 110 may access the comprehensive database storing the readings performed by the one or more IoT devices associated with the structure. The comprehensive database may be broadcasted as a shared ledger to be accessed by the airflow modeling program 110, such that the airflow modeling program 110 may access and/or receive an updated ledger each time a reading is performed by the one or more IoT devices. As will be explained in more detail below, the airflow modeling program 110 may continuously access and/or receive data from the comprehensive database as new readings may be performed by the one or more IoT devices. The airflow modeling program 110 may utilize the comprehensive database in updating a digital twin in real time.

At 204, the airflow modeling program 110 generates a digital twin. The airflow modeling program 110 may generate the digital twin based on the data accessed and/or received with respect to the structure of interest in step 202. A digital twin may be a digital representation of at least an object, entity, and/or system that spans the object, entity, and/or system's lifecycle. The digital twin may be updated using real time data, and may utilize, at least, simulation, machine learning, and/or reasoning in aiding informed decision making. The airflow modeling program 110 may utilize three-dimensional (3D) mapping software to build a digital representation of the structure of interest. The digital representation may be a replica of the physical system of the structure. The digital twin may be utilized by the airflow modeling program in simulating airflow through the structure with respect to one or more infectious diseases.

The airflow modeling program 110 may continuously modify the digital twin based on updates to the comprehensive database, structural data, and/or data provided by the user. The airflow modeling program 110 may update the digital twin in real time based on data received and/or accessed. The digital twin may be stored in a cloud database and/or displayed to the user in the airflow modeling user interface 118. The digital twin displayed by the airflow modeling program 110 may also be updated in real time. For example, the user may adjust the air flow for an office building at the end of the workday. The IoT device associated with the air ventilation system may broadcast the data to the comprehensive database which may be accessed by the airflow modeling program 110 in real time. As will be explained in more detail below, the airflow modeling program 110 may utilize the real time data in simulating the performance of the air ventilation system of the office building based on the adjustments the user made to the air ventilation system.

At 206, the airflow modeling program 110 collects data with respect to one or more infectious diseases. The airflow modeling program 110 may collect data with respect to the one or more infectious diseases from at least one or more publicly available resources and/or from the knowledge corpus (e.g., database 114).

The airflow modeling program 110 may collect data with respect to the one or more infectious diseases based on at least the location of the structure, the time of year, and/or other factors. For example, the airflow modeling program 110 may utilize data scraping and/or web scraping methods in identifying one or more infectious diseases prevalent to the location of the structure.

The user may also identify one or more infectious diseases in the airflow modeling user interface 118 for which the airflow modeling program 110 may collect data. The user may identify one or more infectious diseases based on factors such as, but not limited to, occupants of the structure, past and/or recurring illnesses, novel infectious diseases, amongst other factors in which the user may consider in identifying one or more infectious diseases. For example, the user may identify an office building as the structure of interest. In the past occupants of a particular area within the structure have been caused to miss work with a seasonal influenza. The user may identify the seasonal influenza within the airflow modeling user interface such that the airflow modeling program 110 may collect data with respect to the seasonal influenza identified. The user may provide input provided the airflow modeling program 110 with a description of symptoms, details received from a treating physician, amongst other data such that the airflow modeling program 110 may collect data most relevant to the infectious disease. The airflow modeling program 110 may not receive data with respect to the one or more infectious diseases without receiving consent from all appropriate parties and may not receive and/or access data in violation of any local, state, federal, or international law with respect to data privacy protection.

The airflow modeling program 110 may collect data, such as, but not limited to, transmission medium, basic reproduction number, effective reproductive number, significant clusters, incubation period, amongst other data with respect to the one or more infectious diseases. The airflow modeling program 110 may require a threshold level of data in order to simulate the one or more infectious diseases within the structure of interest. The airflow modeling program 110 may request additional data with respect to the one or more infectious diseases from the user if the data collected is below the threshold level. The data collected for the one or more infectious diseases may enable the airflow modeling program 110 to simulate the one or more infectious diseases within the structure which may be utilized in identifying areas of interest within the structure which may be susceptible to microorganism accumulation. Identifying areas within the structure most susceptible to microorganism accumulation may enable the airflow modeling program 110 to provide the user one or more recommendations which may reduce transmission of the infectious disease between occupants of the structure as will be explained in more detail below.

At 208, the airflow modeling program 110 performs a plurality of simulations of the digital twin based on the data collected for the one or more infectious diseases. The airflow modeling program 110 may simulate varying conditions of the one or more infectious diseases in order to provide one or more recommendations to the user. The airflow modeling program 110 may utilize one or more simulation methods, such as, but not limited to, a Monte Carlo simulation process, agent based simulation model, discrete event simulation model, and/or a system dynamic simulation models, amongst other simulation methods. The airflow modeling program 110 may additionally utilize a statistical program such as IBM's SPSS® (SPSS® and all SPSS-based trademarks are trademarks or registered trademarks of International Business Machines Corporation in the United States, and/or other countries), or Statistical Product and Service Solution, in optimizing the one or more simulation methods.

The airflow modeling program 110 may simulate varying conditions of the one or more infectious diseases by adjusting parameters utilized as input for the one or more simulation models. The airflow modeling 110 may adjust the parameters utilized as input for the one or more simulations models based on the data collected with respect to an infectious disease. For example, the airflow modeling program 110 may adjust the parameters utilized as input for a novel infectious disease for which there may be less available data than a recurring seasonal infectious disease for which there may be more available data. The airflow modeling program 110 may also adjust the parameters based on the location of the structure amongst other data received and/or accessed. For example, the user may identify an office building in Location A as the structure of interest. In this example, the airflow modeling program 110 may adjust the parameters utilized as input for the one or more simulation models based on data representing the employees of the office building as well as historical weather data of Location A.

The airflow modeling program 110 may further utilize the plurality of simulations to generate a heatmap. The heatmap may be integrated with the digital twin such that the digital twin may include one or more indicators as to which areas of the structure's air quality may be harmful based on the simulations of the one or more infectious diseases. The digital twin with the integrated heatmap may be displayed to the user by the airflow modeling program 110 in the airflow modeling user interface 118.

For example, based on the design of the structure and the in-structure air ventilation flow, the airflow modeling program 110 may generate a heatmap displaying particular locations within the structure which may be susceptible to microorganism accumulation and/or higher concentrations for the infectious disease. The airflow modeling program 110 may generate a heatmap of the structure for each of the parameters utilized as input for the one or more simulation models. The airflow modeling program 110 may display the parameters utilized in generating each heatmap with the corresponding digital twin for which the heatmap may be integrated in the airflow modeling user interface 118. The airflow modeling program 110 may utilize indicators such as colors, symbols, and/or other indicators in displaying areas of the structure most susceptible to microorganism accumulation and/or higher concentrations of the infectious disease.

At 210, the airflow modeling program 110 provides one or more recommendations to the user. The airflow modeling program 110 may display the one or more recommendations to the user in the airflow modeling user interface 118. The airflow modeling program 110 may rank the one or more recommendations in the airflow modeling user interface 118 based on at least one or more of, air quality requirements, user preferences, and/or simulated air quality improvement. The recommendations may be based on, for example the particular infectious disease analyzed and the microorganism accumulation within the structure based on the airflow as simulated.

The one or more air quality requirements may be based on local, state, and/or federal regulations for the type of structure and/or the location of the structure as well as internal safety requirements provided to the airflow modeling program 110 by the user. User preferences may include, but are not limited to including, cost of implementation, time frame for implementation, amongst other user preferences. The simulated air quality improvement may be based on the one or more recommendations being simulated using the digital twin. The airflow modeling program 110 may adjust the digital twin for a structural and/or operational recommendation and run the simulation using at least the one or more simulation methods described above with respect to step 208. The airflow modeling program 110 may compare heatmaps for both digital twins. In an embodiment, the airflow modeling program 110 may quantify the air quality improvement based on a comparison of simulated microorganism accumulation for the digital twin with the simulated microorganism accumulation for the digital twin with the one or more recommendations.

The one or more recommendations may include at least structural recommendations and/or operational recommendations. Structural recommendations may include modifications for an existing structure and/or design recommendations for a planned structure. For example, removal of walls, adding windows, installation of one or more IoT devices, amongst other structural recommendations may be made. Operational recommendations may include modifications to processes and/or procedures of the occupants. For example, operational recommendations for a healthcare structure may include, but are not limited to including, recommendations for rotating patient rooms, identifying patient thresholds, room assignment based on diagnosis, sanitation scheduling, adjustments of one or more IoT devices, amongst other operational recommendations.

In an embodiment, the user may enable the airflow modeling program 110 to adjust the environment of the structure utilizing at least the one or more IoT devices associated with the structure. The airflow modeling program 110 may adjust the one or more IoT devices associated with the structure automatically and/or require authorization from the user. The user may authorize automatic adjustments of the one or more IoT devices associated with the structure and/or approve requested adjustments of the one or more IoT devices in the airflow modeling user interface 118.

The airflow modeling program 110 may utilize an intelligent real estate and facilities management solution, such as, but not limited to, IBM TRIRIGA® (IBM TRIRIGA® and all IBM TRIRIGA-based trademarks are trademarks or registered trademarks of International Business Machines Corporation in the United States, and/or other countries), in providing one or more recommendations to the user based on the simulation of the digital twin for the one or more infectious diseases. The airflow modeling program 110 may also utilize the intelligent real estate management solution in at least, monitoring structure occupancy, reducing costs, forecasting maintenance needs, dynamic space planning, indoor mapping, and/or implementation assessments, amongst other recommendations. The intelligent real estate and facilities management solution may enable the user to interactively engage with the airflow modeling program 110 in the airflow modeling user interface. For example, allowing the user to make structural adjustments and simulate the air quality improvement prior to making structural changes.

It may be appreciated that FIG. 2 provides only an illustration of one embodiment and do not imply any limitations with regard to how different embodiments may be implemented. Many modifications to the depicted embodiment(s) may be made based on design and implementation requirements.

FIG. 3 is a block diagram 900 of internal and external components of computers depicted in FIG. 1 in accordance with an illustrative embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902 a, b and external components 904 a, b illustrated in FIG. 3. Each of the sets of internal components 902 a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the airflow modeling program 110a in client computer 102, and the airflow modeling program 110b in network server 112, may be stored on one or more computer-readable tangible storage devices 916 for execution by one or more processors 906 via one or more RAMs 908 (which typically include cache memory). In the embodiment illustrated in FIG. 3, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the airflow modeling program 110a and 110b can be stored on one or more of the respective portable computer-readable tangible storage devices 920, read via the respective R/W drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The software program 108 and the airflow modeling program 110a in client computer 102 and the airflow modeling program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the airflow modeling program 110a in client computer 102 and the airflow modeling program 110b in network server computer 112 are loaded into the respective hard drive 916. The network may comprise copper wires, optical fibers, wireless transmission, routers, firewalls, switches, gateway computers and/or edge servers.

Each of the sets of external components 904 a, b can include a computer display monitor 924, a keyboard 926, and a computer mouse 928. External components 904 a, b can also include touch screens, virtual keyboards, touch pads, pointing devices, and other human interface devices. Each of the sets of internal components 902 a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

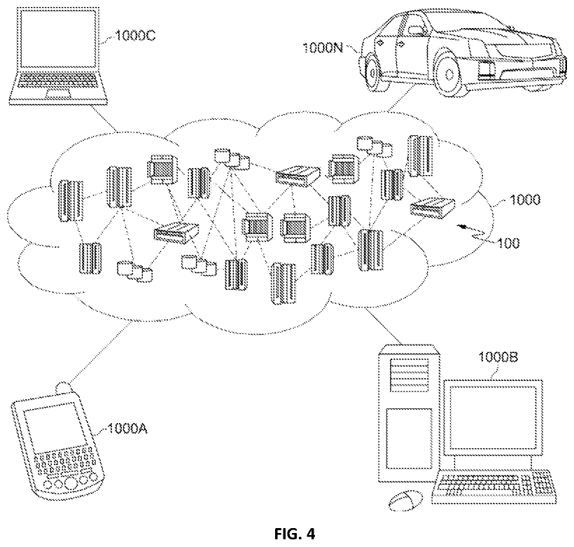

Referring now to FIG. 4, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 4 are intended to be illustrative only and that computing nodes 100 and cloud computing environment 1000 can communicate with any type of computerized device over any type of network and/or network addressable connection (e.g., using a web browser).

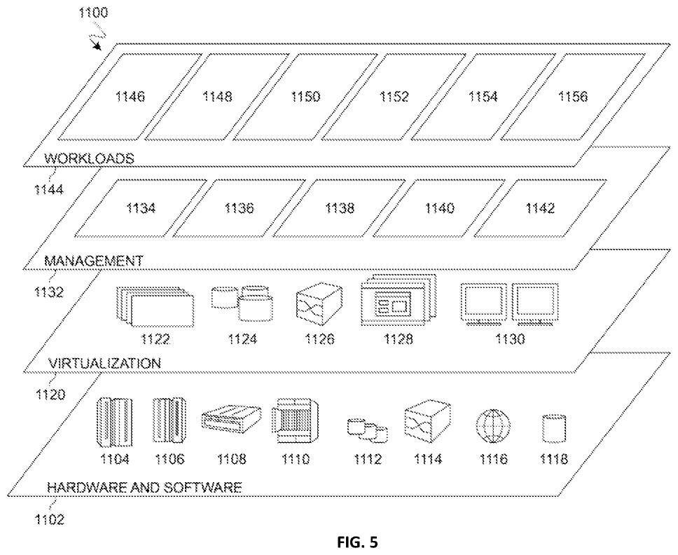

Referring now to FIG. 5, a set of functional abstraction layers 1100 provided by cloud computing environment 1000 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and airflow modeling 1156. An airflow modeling program 110a, 110b provides a way to provide one or more recommendations to a client based on at least a performance of a digital twin in a plurality of simulations for one or more infectious diseases.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

The present disclosure shall not be construed as to violate or encourage the violation of any local, state, federal, or international law with respect to privacy protection.

What is claimed is:

1. A method for airflow modeling, the method comprising:
generating a digital twin for a structure of interest identified by a user;
collecting data for one or more infectious diseases;
performing a plurality of simulations of the digital twin for the one or more infectious diseases; and
providing one or more recommendations to the user based on at least a performance of the digital twin in the plurality of simulations for the one or more infectious diseases.

2. The method of claim 1, further comprising:
generating a heatmap based on the plurality of simulations of the digital twin.

3. The method of claim 2, wherein the heatmap is integrated with the digital twin such that the digital twin includes one or more indicators as to which areas of the structure of interest are most susceptible to microorganism accumulation.

4. The method of claim 1, wherein the digital twin is generated based on data received from one or more IoT devices associated with the structure of interest.

5. The method of claim 4, further comprising:
adjusting settings for the one or more IoT devices associated with the structure of interest based on the plurality of simulations.

6. The method of claim 1, wherein the one or more recommendations are provided to the user in an airflow modeling user interface.

7. The method of claim 1, wherein the one or more recommendations are based on at least, one or more of, an air quality requirements, user preferences, or a simulated air quality improvement.

8. A computer system for airflow modeling, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
generating a digital twin for a structure of interest identified by a user;
collecting data for one or more infectious diseases;
performing a plurality of simulations of the digital twin for the one or more infectious diseases; and
providing one or more recommendations to the user based on at least a performance of the digital twin in the plurality of simulations for the one or more infectious diseases.

9. The computer system of claim 8, further comprising:
generating a heatmap based on the plurality of simulations of the digital twin.

10. The computer system of claim 9, wherein the heatmap is integrated with the digital twin such that the digital twin includes one or more indicators as to which areas of the structure of interest are most susceptible to microorganism accumulation.

11. The computer system of claim 8, wherein the digital twin is generated based on data received from one or more IoT devices associated with the structure of interest.

12. The computer system of claim 11, further comprising:
adjusting settings for the one or more IoT devices associated with the structure of interest based on the plurality of simulations.

13. The computer system of claim 8, wherein the one or more recommendations are provided to the user in an airflow modeling user interface.

14. The computer system of claim 8, wherein the one or more recommendations are based on at least, one or more of, an air quality requirements, user preferences, or a simulated air quality improvement.

15. A computer program product for airflow modeling, comprising:
one or more non-transitory computer-readable storage media and program instructions stored on at least one of the one or more tangible storage media, the program instructions executable by a processor to cause the processor to perform a method comprising:
generating a digital twin for a structure of interest identified by a user;
collecting data for one or more infectious diseases;
performing a plurality of simulations of the digital twin for the one or more infectious diseases; and
providing one or more recommendations to the user based on at least a performance of the digital twin in the plurality of simulations for the one or more infectious diseases.

16. The computer program product of claim 15, further comprising:
generating a heatmap based on the plurality of simulations of the digital twin.

17. The computer program product of claim 16, wherein the heatmap is integrated with the digital twin such that the digital twin includes one or more indicators as to which areas of the structure of interest are most susceptible to microorganism accumulation.

18. The computer program product of claim 15, wherein the digital twin is generated based on data received from one or more IoT devices associated with the structure of interest.

19. The computer program product of claim 18, further comprising:
adjusting settings for the one or more IoT devices associated with the structure of interest based on the plurality of simulations.

20. The computer program product of claim 15, wherein the one or more recommendations are provided to the user in an airflow modeling user interface.
